(19) United States
(12) Patent Application Publication
(10) Pub. No.: US 2021/0338214 A1
(43) Pub. Date: Nov. 4, 2021

(54) A DEVICE FOR ENDOSCOPIC EXAMINATION CUM SAMPLE COLLECTION AND METHOD THEREOF

(71) Applicants: Deeksha Pandey, Manipal, Karnataka (IN); Rishi Sethi, Lucknow, Uttar Pradesh (IN)

(72) Inventors: Deeksha Pandey, Manipal, Karnataka (IN); Rishi Sethi, Lucknow, Uttar Pradesh (IN); Abhishek Verma, Barabanki, Uttar Pradesh (IN); Janakarajan Ramkumar, Kanpur (IN); Virender Singh, Kanpur (IN); Adarsh Kumar, Kanpur (IN)

(21) Appl. No.: 17/272,006

(22) PCT Filed: Aug. 30, 2019
(86) PCT No.: PCT/IB2019/057322

§ 371 Date: Feb. 26, 2021

(30) Foreign Application Priority Data
Aug. 30, 2018 (IN) 201811032508

(51) Int. Cl. A61B 10/02; A61B 1/05; A61B 1/00; A61B 1/015; A61B 10/04; A61B 1/06; A61B 1/303

(52) U.S. Cl. CPC A61B 10/0291; A61B 1/05; A61B 1/00087; A61B 1/00101; A61B 1/00082; A61B 1/00066; A61B 1/227; A61B 1/00032; A61B 10/04; A61B 1/0684; A61B 1/303; A61B 2010/0216; A61B 1/015

(57) ABSTRACT

The present invention provides an endoscopic device for examination cum sample collection of an orifice of a user's body. In particular, the device is useful for vaginal and cervical examination. The device comprises a camera attached on the probe along with a radial luminous system and cytobrush for sample collection attached within the probe. A latex balloon and air flow directly through port are provided to inflate the vaginal cavity. The invention also provides the method for sample collection and supervision through controlled camera.

### FIELD OF THE INVENTION

The present Invention relates to a device and method endoscopic examination cum sample collection. Particularly, the device is an integrated device performing the dual purpose of routine gynaecological examination and cervical cancer screening.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art.

Speculum examination is an integral part of Gynecological evaluation wherein the lower genital tract (cervix and vagina) is visualized for any abnormality. The gynaecological examination is required to detect sexually transmitted infections, benign lesions of lower genital tract, abnormalities and cancers of cervix and vagina. Almost every woman attending Gynecology outpatient (OPD) undergoes this test. A metal (rarely a disposable plastic) instrument called Cusco's speculum is inserted in the vagina to open up the vaginal canal (a potential cavity), so as the vaginal walls and cervix can be visualized.

The importance of visualizing the cervix lies in the fact that it is the site of many pathologies/diseases including cervical cancer. When coupled with collection of cytology samples from the surface of cervix, this examination facilitates screening of early cervical cancer or its pre-cancer stages.

Cervical screening is the process of recognizing abnormal tissue or cells in the cervix at the precancerous stages before cervical malignancy develops. There are various types of tests for Cervical Cancer Screening. Pap smear (Cytological screening) is still the most commonly performed screening test owing to its ease of performance, simplicity and cost effectiveness. Other important indications of performing speculum examination is to identify sexually transmitted infections, polyps, erosions and other benign and malignant pathologies of vagina and cervix.

Although extremely common and important, this procedure is the most uncomfortable part of entire gynecological examination. Thus, there is the need of a universal solution which can reduce the discomfort and pain during this procedure.

The survey says that the cervical cancer screening global market is poised to reach $22 billion by 2020 from ˜$15 billion in 2014, at an estimated CAGR of 7.0% from 2015 to 2020. Asia Pacific is expected to emerge as the fastest growing region with a CAGR of 9.6% over the forecast period.

Cervical cancer screening market in India is in still infancy stage. Currently the market is highly unorganized in the nature as most of the screening tests are performed in hospitals or under public programmes.

At present, Cusco's or Sims speculum/s are being used by the gynaecologist to examine women who present to Gynaecology OPD. In a few settings, a plastic version of the same which is disposable, thus more costly is used but is none the less almost equally uncomfortable. Cervical cancer screening is usually combined with this examination.

At present there is no device in the market which can both perform thorough lower genital tract examination as well as collect cervical cytology sample in an easy, painless, focussed and patient friendly way. Following are few of the devices present in the market being currently used for the examination:
a) Metal Speculum, useful for Imaging and diagnosing of abnormal cells in a target tissue. However, this is painful and discomfort in use.
b) Pocket colposcopy: This is a device which is speculum free and painless, being used for cervix imaging. However, the device fails for the purpose of sample collection, detection of pathologies other than cervical cancer. It does not perform vaginal wall examination.
c) Pocket colposcopy: This is also speculum free, painless, useful for cervix imaging. However, the device fails to collect the sample, does not detect pathologies other than cervical cancer and it does not have provision for vaginal wall examination.
d) Helvoet's Delphi screener: This is speculum free, painless, facilitates cell collection. However, it does not perform cervix imaging, no vaginal wall detection and also does not detect pathologies other than cervical cancer. The device cannot be reused as well.
e) Rovers Delphi screener: It employs lavage method for cell collection without scraping. This is speculum free, quick and comfortable. However, it does not perform cervix imaging and cannot be reused as well.

The existing products present in the market, as discussed above, suffer/s with the drawback of being painful, uncomfortable, not being able to be reused along with not being able to collect the sample, detect pathologies other than cervical cancer, no vaginal wall examination. There is also no such device in the market, which combines the feature of the routine gynaecological examination and cervical cancer screening. Hence, there is a need of a user friendly, minimally invasive and an efficient device useful for vaginal and cervical examination.

### OBJECT/S OF PRESENT INVENTION

A primary object of the present invention is to overcome the drawbacks associated with the prior art.

Yet another object of the present invention is to provide a user friendly, minimally invasive and an efficient device useful for vaginal and cervical examination.

Yet another object of the present invention is to provide an efficient device that provides the opportunity of visualizing the complete lower genital tract for anatomical/pathophysiological abnormalities and incorporates this with cytological sampling for cervical cancer screening.

Yet another object of the present invention is to provide an efficient device that helps in making a visual diagnosis of various discharges and lesions as well along with collecting exfoliative cytology sample for cervical cancer screening in the most optimal way.

Yet another object of the present invention is to provide a reusable and sterilization compatible device.

Yet another object of the present invention is to provide a portable and easy hand holding device.

Yet another object of the present innovation is to digitalize the imaging and combine it with blue tooth transfer technology for discussion/second opinion with experts sitting far away from the place where the examination is being carried out.

### SUMMARY OF THE INVENTION

In an aspect, there is provided an endoscopic device for examination cum sample collection from orifice of a user's body comprising:
a) atleast a movable probe with a proximal end adapted to be inserted in said orifice and a distal end configured to be outside said orifice;
   said proximal end comprises atleast a balloon like structure to be inflated/deflated positioned on its terminal/side, atleast a camera with a radial luminescence system for visual inspection, atleast a detachable cytobrush extending from a structured port to the exterior of said end useful for sample collection, plurality of ports to pass air to said orifice for regulated inflation/deflation of said orifice;
   while said distal end of said probe is connected to a grooved handle;
   where said balloon like structure prevents leakage of air from said orifice;
b) a reusable grooved handle, provided with plurality of switches/buttons to control the functioning of said device, adapted to be positioned outside said orifice, comprising a hollow frame with atleast a first and a second end where the first end is connected with the distal end of said probe through a detachable connecting means;
   said hollow frame is adapted to accommodate electronic components which functions in conjunction to said plurality of switches for achieving the desired function/working of the device, comprising atleast a controlled power source unit for supplying controlled power to a motor unit which further connects to drive plurality of pump/s; wherein atleast a pump is configured to supply air to said balloon like structure through airtube/s connection which extends through said probe internally to be connected to the proximal end of said probe;
   while atleast a pump is configured to supply air to said orifice through airtube/s connection which extends through said probe internally to be connected to the proximal end of said probe;
c) atleast an output port positioned at the second end of said grooved handle comprising monitoring unit to assess the optical/electrical signal received from said camera, facilitating the examination/sample collection from said orifice.

In another aspect, there is provided a method of operation of device described above. The method comprises the steps of:
a) inserting the probe of the device into said orifice for examination/sample collection followed by activating the probe camera by switching it ON through switches placed on said handle;
b) providing a controlled inflation/deflation by the combined action of activating balloon like structure along with passing the pressurised air from pumps through plurality of ports to said orifice where said pump is activated through said switch/es;
c) examining/assessing the internal visual view of said orifice through camera connected with the monitoring unit;
d) optionally inserting the cytobrush to the probe through the port provided in the frame under the supervision of controlled camera for collecting the sample.

### BRIEF DESCRIPTION ABOUT DRAWINGS

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings in which:

FIG. 1: provides an exterior perspective view of device embodying the present invention

FIG. 2; provides the perspectives view of (i) front (ii) back view of probe of device as an embodiment of the present invention

FIG. 3: provides the perspective view (i) exterior (ii) interior view of grooved handle comprising hollow frame as an embodiment of the present invention

FIG. 4: provides the perspective of the interior accommodating components of hollow frame as an exemplary embodiment of the present invention

FIG. 5: provides the perspective view (i) connection between probe and first end of frame and (ii) isomeric view of the first end of hollow frame as an embodiment of the present invention

FIG. 6: provides the perspective view of connection between probe and frame of device as an embodiment of the invention.

FIG. 7: provides the perspective view of air tube connection with probe and electronic connection of probe camera of device embodying the present invention

FIG. 8: provides the electromyogram study on holding devices or performing a task as an exemplary embodiment of the present invention.

FIG. 9: provides the plot of time versus pressure plot to characterization of the balloon inflating motors of device.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof. Throughout the patent specification, a convention employed is that in the appended drawings, like numerals denote like components.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

In an aspect of the present invention, there is provided an endoscopic device for examination cum sample collection of an orifice of a user's body. In particular, the device is useful for vaginal and cervical examination. The device will be useful in routine evaluation of lower genital tract with the help of a patient friendly endoscope. The device is an integrated device that provides the opportunity of visualizing the complete lower genital tract for anatomical/pathophysiological abnormalities (magnified vision) and incorporates this with cytological sampling for cervical cancer screening.

The device has application in visual inspection of vaginal introitus walls for health/infection concerns (Routine gynecology checks) using camera attached on the probe along with a radial luminous system along the periphery of the camera. It can also used for cervical cancer screening using camera attached on the probe along with a radial luminous system along the periphery of the camera and Vaginal biopsy using sample collection using cyto-brush attached within probe. The provided device can inflate vaginal introitus cavity that collapse on itself (soft tissues) using a latex balloon. Device can inflate vaginal introitus cavity that collapse on itself (soft tissues) using air directly into cavity.

In an embodiment, the endoscopic device for examination cum sample collection of an orifice of a user's body comprises following components:
a) atleast a movable probe with a proximal end adapted to be inserted in said orifice and a distal end configured to be outside said orifice;
   said proximal end comprises atleast a balloon like structure to be inflated/deflated positioned on its terminal/side, atleast a camera with a radial luminescence system for visual inspection, atleast a detachable cytobrush extending from a structured port to the exterior of said end useful for sample collection, plurality of ports to pass air to said orifice for regulated inflation/deflation of said orifice;
   while said distal end of said probe is connected to a grooved handle;
   where said balloon like structure prevents leakage of air from said orifice;
b) a reusable grooved handle, provided with plurality of switches/buttons to control the functioning of said device, adapted to be positioned outside said orifice, comprising a hollow frame with atleast a first and a second end where the first end is connected with the distal end of said probe through a detachable connecting means;
   said hollow frame is adapted to accommodate electronic components which functions in conjunction to said plurality of switches for achieving the desired function/working of the device, comprising atleast a controlled power source unit for supplying controlled power to a motor unit which further connects to drive plurality of pump/s; wherein atleast a pump is configured to supply air to said balloon like structure through airtube/s connection which extends through said probe internally to be connected to the proximal end of said probe;
   while atleast a pump is configured to supply air to said orifice through airtube/s connection which extends through said probe internally to be connected to the proximal end of said probe;
c) atleast an output port positioned at the second end of said grooved handle comprising monitoring unit to assess the optical/electrical signal received from said camera, facilitating the examination/sample collection from said orifice.
In an embodiment, the first end of the hollow body frame comprises a void to accommodate the probe with said frame.
In an embodiment, the void comprises plurality of ports for connecting pressurized air supply from the pump into the probe, atleast a port for camera wiring, atleast a slot for removing the cytobrush along with probe without contaminating handle.
In an embodiment, the cytobrush is to be controlled from the handle end where the cytobrush is positioned through structured port of said probe that passes through a port below the camera in said probe on its proximal end for achieving movement in said orifice.
In an embodiment, the motor unit operates through said buttons placed on the top of the handle thus actuating said plurality of pumps for regulating inflation/deflation of orifice.
In an embodiment, the pump unit inside the hollow body frame provides pressured air supply to said orifice and the balloon through air tube/s.
In an embodiment, the orifice comprises vagina, mouth, ear, nose, anus.
In an embodiment, the power supply unit comprises atleast a battery and a voltage control unit and connection/s thereof.
In an embodiment, the probe camera comprises camera with LED illumination being regulated and monitored by output monitor unit connected through optical cable.
In an embodiment, the monitoring unit comprises mobile, computer, smart TV and like.

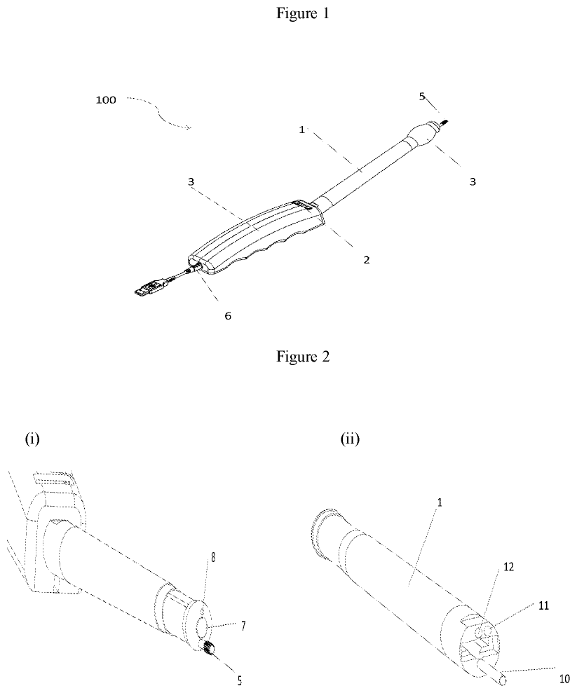

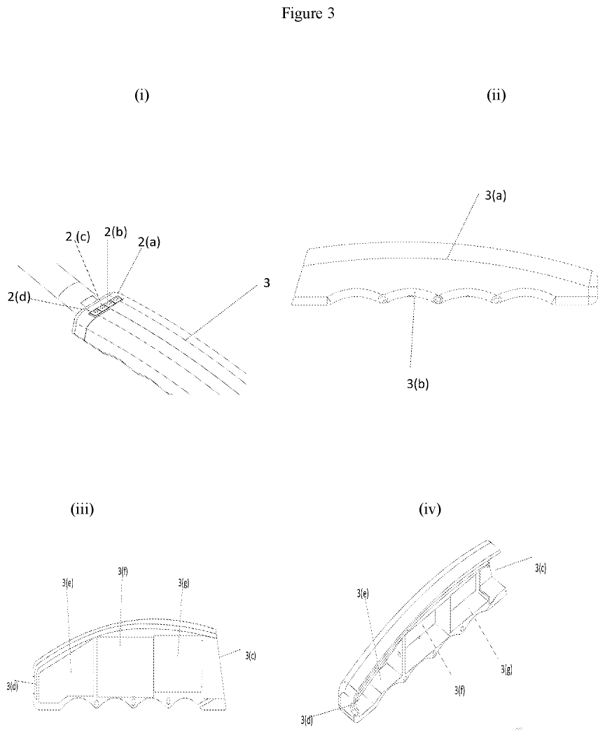

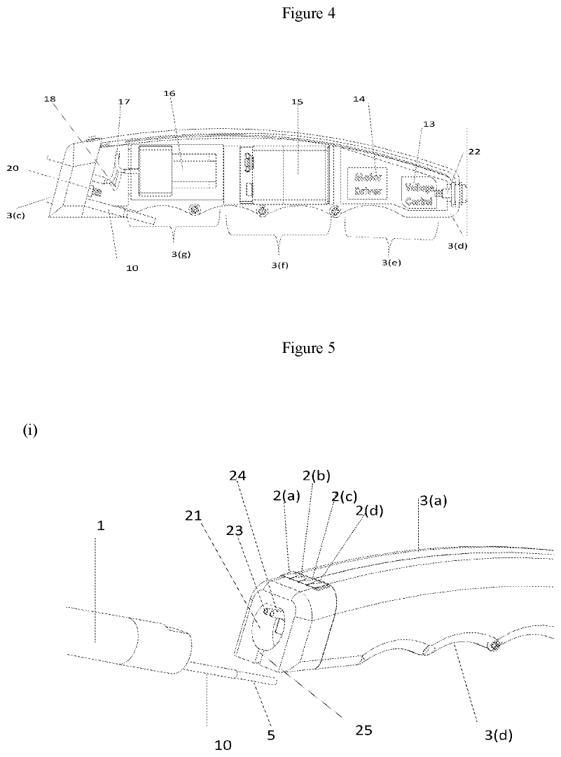

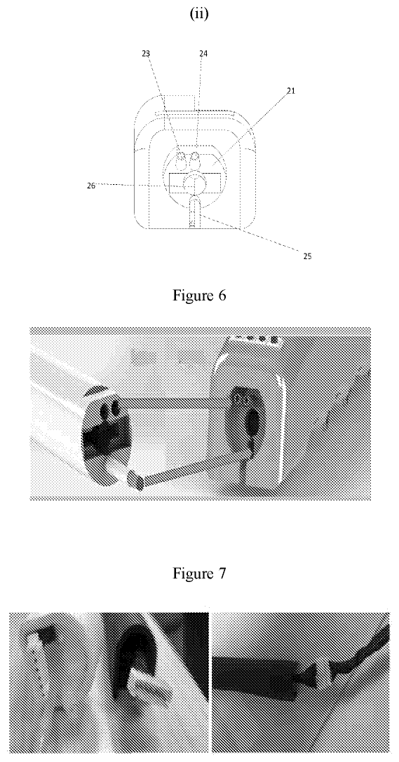

The Invention is further described with the help of following non-limiting embodiments in conjunction with figures like FIGS. 1, 2 and 3 and 4,5 and 6.
FIG. 1 illustrates a perspective exterior view of the device 100 as accordance to the exemplary embodiment of the present invention. The device comprises a probe (1), a grooved handle 2 a body frame and output port (6).
FIGS. 2(i) and 2(ii) illustrate the perspective view of probe as an exemplary embodiment and probe (1) having a proximal end (FIG. 2(i)) and a distal end (FIG. 2(ii)) where the proximal end comprising a camera (7) with a radial luminescence system for visual inspection, a removable cytobrush (5) for sample collection, ports for air flow (8) and a balloon (4) adapted to inflate introitus cavity and wherein the said balloon connected with outer peripheral surface of the proximal end of the said probe (1). Referring to the FIG. 2(u) provides the perspective view of distal end of the probe (1) and the cavity 11 an 12 provided to fix the connecting element for air flow to balloon and ports to pass air to said orifice for regulated inflation/deflation of the cavity of said orifice and small extended cylindrical cover or sleeve 10 is provided to cover cyto-brush. In an exemplary embodiment the maximum diameter of probe not more than 17 cm for patient comfort Orientation of probe (1) compliments anatomy of vaginal Introitus. Use and throw probe part with reusable handle which contains all the controllers and electronics for sterilization compliance.
FIG. 3(i) to (iv) provides the exterior and interior perspective view of grooved handle and comprising of a hollow frame as accordance to the exemplary embodiment of the present invention. The grooved handle (3) has a hollow body frame and frame has a top (3(a)) and a bottom surface (3(b)) and the bottom surface comprises grooves for tightly holding the device. The frame is having a first (3(c)) and a second end (3(d)) where the first end connected with the distal end of the said probe (1) through a detachable connecting means. The hollow frame comprises plurality of space section (3(e), 3(f) and 3(g)) inside the hollow frame of grooved handle (3). Referring to FIG. 4 the space provided in the hollow frame accommodating a controlled voltage source (13) connected through the power source (15) and provides the controlled power to a pump unit (16) connected through a motor (14) operated through plurality of switches (2(a),2(b) 2(c) and 2(d)) present on the top surface (3(a) of the frame to control inflation and deflation for probe balloon (4) and cavity (8) connecting through a air tubes (18) and (19) by the pump unit.
The provision for attaching/detaching of probe has been provided by creating a void (21) (FIG. 5) in hollow body frame which accommodating the probe (1) with the said frame and wherein the said void (21) comprises a pair of port 23 and 24 for connecting pressurized air supply from the pump into the probe, a port for camera wiring (26) and a slot (25) for removing the cytobrush through the handle along with probe (1).
In an exemplary embodiment, connection of camera is provided using a 6-Pin plug that is detached from the probe when probe is removed from the frame part. A partial representation of this plug is presented in FIGS. 6 and 7. FIG. 6 represent air tube plugging between motor pump and balloon/cavity while FIG. 7 represent electronic connection of camera.
In an exemplary embodiment for serving the purpose of portability and ergonomic design the body frame act as a handle and made to accommodate all the electronics and at the same time connected with the probe using male and female conical tube connectors. From FIG. 4 and FIG. 5 it can be observed that although the cytobrush is inserted from behind the handle, it is still protected by an extended sleeve (10) on the probe. Therefore, when probe is detached from the handle, this extended sleeve (10) slides gently through a slot from the handle without contaminating any part of the handle.
In preferred exemplary embodiment FIG. 1 provides an output port (6) connected to the second end of the said hollow body frame (3(d)) and adapted to visualize the optical/electrical signal coming from the probe camera (7) to a system comprising monitoring unit visualize the examination. In an embodiment the system connecting with the monitoring device comprises mobile device android system or IOS system, or computing device with monitor screen or smart TV screen. In a preferred embodiment the output port (6) connected directly to an Android device or a Windows operating system.

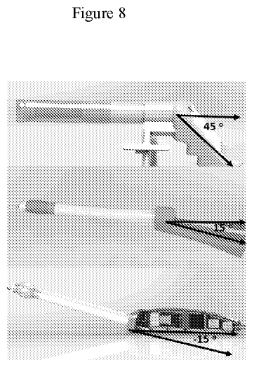

In an exemplary embodiment pump unit (16) inside the hollow body frame comprises two pumps and provides the pressurized air supply to the ports for air flow (8) and the balloon through air tubes (18,19) connecting the pump. One pump provides the pressurised ports for air e e and other to the balloon by connecting air tubes.
In an embodiment cytobrush has handle connecting with slot provided in the body frame and covered with sleeve (10) presented in distal end and movement in said cytobrush provided manually through the handle of said cytobrush under the body frame. Cytobrush does not come in contact with grooved handle because of the design of the cytobrush sleeve into the probe,
In an exemplary embodiment the orientation of the probe can be observed to be evolving based on ergonomic principles. An illustrative FIG. 8 represents the electromyogram study on holding devices or performing a task. It is evaluated from qualitative and quantitative ergonomic analyses that the angles between −15 o and +15o are best suited for design of handheld devices, specifically medical diagnostic devices that are needed to be used for prolonged time (more than 20 minutes) where stability is major concern.

Another aspect of the present invention provides a method of operation of device for examination of an orifice.

In an embodiment, the method comprises following steps:
a) inserting the probe of the device into said orifice for examination/sample collection followed by activating the probe camera by switching it ON through switches placed on said handle;
b) providing a controlled inflation/deflation by the combined action of activating balloon like structure along with passing the pressurised air from pumps through plurality of ports to said orifice where said pump is activated through said switch/es;
c) examining/assessing the internal visual view of said orifice through camera connected with the monitoring unit;
d) optionally inserting the cytobrush to the probe through the port provided in the frame under the supervision of controlled camera for collecting the sample.

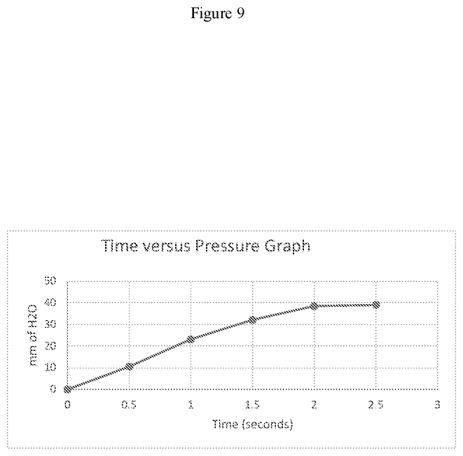

In an exemplary embodiment, the device provided in the in invention is based on a size of the electric motor small enough to be accommodated into the frame and robust enough to inflate against pressure of self-closed mucosal membrane of vagina which is about 10-12 cm of H₂O while in a resting position. Two independent motors are used for inflating balloon and cavity directly. Each motor (commercially available in the market) is rated 130 milliampere, 6 Volts and generating air at 407 cm of H₂O. The characterization of the balloon inflating motors is done plotting time versus pressure plot, as shown in FIG. 9. The inflated latex balloon is made up of latex with thickness of 0.3 mm. FIG. 10 provides the time verses pressure plot for inflating 0.3 mm thickness latex balloon at 6 volts power.

1. An endoscopic device for examination cum sample collection from orifice of a user's body comprising:
a) atleast a movable probe with a proximal end adapted to be inserted in said orifice and a distal end configured to be outside said orifice;
said proximal end comprises atleast a balloon like structure to be inflated/deflated positioned on its terminal/side, atleast a camera with a radial luminescence system for visual inspection, atleast a detachable cytobrush extending from a structured port to the exterior of said end useful for sample collection, plurality of ports to pass air to said orifice for regulated inflation/deflation of said orifice;
while said distal end of said probe is connected to a grooved handle;
where said balloon like structure prevents leakage of air from said orifice;
b) a reusable grooved handle, provided with plurality of switches/buttons to control the functioning of said device, adapted to be positioned outside said orifice, comprising a hollow frame with atleast a first and a second end where the first end is connected with the distal end of said probe through a detachable connecting means;
said hollow frame is adapted to accommodate electronic components which functions in conjunction to said plurality of switches for achieving the desired function/working of the device, comprising atleast a controlled power source unit for supplying controlled power to a motor unit which further connects to drive plurality of pump/s;
wherein atleast a pump is configured to supply air to said balloon like structure through airtube/s connection which extends through said probe internally to be connected to the proximal end of said probe;
while atleast a pump is configured to supply air to said orifice through airtube/s connection which extends through said probe internally to be connected to the proximal end of said probe;
c) atleast an output port positioned at the second end of said grooved handle comprising monitoring unit to assess the optical/electrical signal received from said camera, facilitating the examination/sample collection from said orifice.

2. The device as claimed in claim 1, wherein the first end of the hollow body frame comprises a void to accommodate the probe with said frame.

3. The device as claimed in claim 2, wherein said void comprises plurality of ports for connecting pressurized air supply from the pump into the probe, atleast a port for camera wiring, atleast a slot for removing the cytobrush along with probe without contaminating handle.

4. The device as claimed in claim 1, wherein said cytobrush is to be controlled from the handle end where the cytobrush is positioned through structured port of said probe that passes through a port below the camera in said probe on its proximal end for achieving movement in said orifice.

5. The device as claimed in claim 1, wherein said motor unit operates through said buttons placed on the top of the handle thus actuating said plurality of pumps for regulating inflation/deflation of orifice.

6. The device as claimed in claim 1, wherein the pump unit inside the hollow body frame provides pressured air supply to said orifice and the balloon through air tube/s.

7. The device as claimed in claim 1, wherein said orifice comprises vagina, mouth, ear, nose, anus.

8. The device as claimed in claim 1, wherein said power supply unit comprises atleast a battery and a voltage control unit and connection/s thereof.

9. The device as claimed in claim 1, wherein the probe camera comprises camera with LED illumination being regulated and monitored by output monitor unit connected through optical cable.

10. The device as claimed in claim 1, wherein the monitoring unit comprises mobile, computer, smart TV and like.

11. A method of operation of device as claimed in claim 1, comprising the steps of:
a) inserting the probe of the device into said orifice for examination/sample collection followed by activating the probe camera by switching it ON through switches placed on said handle;
b) providing a controlled inflation/deflation by the combined action of activating balloon like structure along with passing the pressurised air from pumps through plurality of ports to said orifice where said pump is activated through said switch/es;
c) examining/assessing the internal visual view of said orifice through camera connected with the monitoring unit;
d) optionally inserting the cytobrush to the probe through the port provided in the frame under the supervision of controlled camera for collecting the sample.
